SEQUENCE LISTING (5 sequences)

SEQ ID NO: 1 - protein, 21 aa, Artificial
  A chain from examples 1-4
GIVEQCCHSICSLYQLENYCN

SEQ ID NO: 2 - protein, 29 aa, Artificial
  B chain from example 1
FVNQHLCGSHLVEALYLVCGERGFNYTPK

SEQ ID NO: 3 - protein, 29 aa, Artificial
  B chain from example 2
FVNQHLCGSHLVEALYLVCGERGFNYEPK

SEQ ID NO: 4 - protein, 29 aa, Artificial
  B chain from example 3
FVNQHLCGSHLVEALYLVCGERGFHYDPK

SEQ ID NO: 5 - protein, 29 aa, Artificial
  B chain from example 4
FVNQHLCGSHLVEALYLVCGERGFHYDPK

(19) United States
(12) Patent Application Publication
(10) Pub. No.: US 2011/0294729 A1
(43) Pub. Date: Dec. 1, 2011

(54) Novel Insulin Analogues

(75) Inventors: Carsten Enggaard Stidsen, Soborg (DK); Tine Glendorf, Hillerod (DK); Thomas Börglum Kjeldsen, Virum (DK)

(73) Assignee: Novo Nordisk A/S, Bagsvaerd (DK)

(21) Appl. No.: 13/133,465

(22) PCT Filed: Dec. 3, 2009
(86) PCT No.: PCT/EP2009/066335

§ 371 Date: Aug. 5, 2011

(30) Foreign Application Priority Data
Dec. 9, 2008 (EP) 08171086.5
Mar. 13, 2009 (EP) PCT EP009053017
May 25, 2009 (EP) 09161014.7
May 27, 2009 (EP) 09161232.5

(51) Int. Cl. A61K 38/28; A61P 3/10; C07K 14/62

(52) U.S. Cl. 514/6.2; 530/303

(57) ABSTRACT

Insulin analogues wherein the B25 amino acid residue is His or Asn with the proviso that if the B25 amino acid residue is His, then the B27 amino acid residue is Asp or Glu and the A14 amino acid residue is different from Glu, have liver preferential actions.

### FIELD OF THIS INVENTION

The present invention relates to novel insulin analogues.

### BACKGROUND OF THIS INVENTION

Insulin is a pancreatic hormone involved in the regulation of blood-glucose concentrations in humans as well as having a role in protein and lipid metabolism. Insulin was discovered in the early 20'iest.

Early commercial insulins (porcine and bovine insulins) were obtained by extraction from bovine or porcine pancreases followed by purification. In the 80'iest, human insulin was put on the market. One way of preparing human insulin was by recombinant DNA technology. Soon, the recombinant DNA technology was developed further and many different insulin analogues were prepared and some are now on the marked. In the last century, many different insulin products have been on the marked, also including insulin zinc suspensions, insulin complexes, e.g. with protamine sulphate, and insulin derivatives, e.g. acylated insulins.

Nearly every society on earth is affected by diabetes—one of the most pressing health crises facing humanity today. Currently, more than 246 million people worldwide are living with the disease, a number that is expected to explode to 380 million within 19 years.

The insulin receptor (IR) is expressed as two isoforms, IR-A and IR-B, originating from alternative splicing of the insulin receptor mRNA involving exon 11 of the IR gene. The IR-A a-subunit lacks the C-terminal amino acid sequence encoded for by exon 11.

The two isoforms of the insulin receptor are differentially expressed. The B-isoform is dominantly (90%) expressed in the liver, but it is otherwise widely expressed in most tissues together with the A-isoform (˜50/50).

In normal individuals as well as in diabetic individuals, the liver produces glucose in order to avoid hypoglycemia. In type 2 diabetes patients, however, the regulation of hepatic glucose output is poorly controlled and is increased, and may be doubled after an over-night fast. Furthermore, sub-cutaneous insulin administration delivers insulin to the peripheral tissues before the liver, while under normal physiological conditions, insulin is delivered directly to the liver from the pancreas, such that the insulin concentration exposed to the liver is 3-4 times higher than in peripheral tissues such as fat and muscle. Diabetic patients would benefit from being treated with a liver-preferential insulin analogue, thereby effectively reducing hepatic glucose output with little effect in the peripheral tissues, leading to improved glucose control, reduced risk of hypoglycaemia, less weight gain, better lipid profiles and cardio-protective effects. One way of pursuing that is to develop insulin analogues having preference for the B-isoform of the insulin receptor. However, such analogues are currently not known. Human insulin reportedly has a slightly higher affinity for the insulin receptor A-isoform than for the B-isoform, vide the EMBO Journal 9 (1990), 2409-13.

There is also a temporal dimension to the expression of the insulin receptor isoforms. IR-A is mainly expressed during fetal development, after which differentiating cells upregulate their expression of IR-B. IR-A is upregulated in several tumors and cancer cell lines, including breast and colon cancers, where it mediates some of the proliferative effects of IGF-II, which has 40-50 fold higher affinity for IR-A than for IR-B.

According to WO 90/01038, His^{B25} human insulin; His^{B25}, Asp^{B28}, desB30 human insulin and His^{B25} human insulin B30 amide have high biological activity. According to EP 837,072 A2, His^{B25}, des B26, desB30 human insulin has low ability to associate in solution. There is no mentioning of hepato activity in these publications. According to WO 2008/034881, some A14E, B25H and B25H, B27T insulin analogues exhibit resistance towards protease. In WO 00/69901, there are described “IA proteins with amino acid sequences that have at least about 1-20 amino acid substitutions as compared to human insulin”, specifically 6 amino acids in FIG. 5A. According to claim 9 in WO 00/69901, the amino acid N (Asn) is one of the possible substitutions in position B25 and since claim 9 refers only to claim 8, the B25N substitution is only mentioned in passing for IA proteins comprising at least 5 substitutions.

In U.S. Pat. No. 5,446,020, there are described insulin analogues which are modified at amino residues A12, A14, A15 and A19. WO 90/12814 discloses hepato-specific insulin analogues which are substituted at the A13, A14, A15, A19 and B16 positions.

### OBJECTS OF THIS INVENTION

The object of this invention is to overcome or ameliorate at least one of the disadvantages of the prior art, or to provide a useful alternative.

One aspect of this invention relates to the furnishing of insulin analogues with higher affinity for the B-isoform than for the A-isoform of the insulin receptor.

Another aspect of this invention relates to the furnishing of insulin analogues giving improved glucose control.

Another aspect of this invention relates to the furnishing of insulin analogues offering reduced risk of hypoglycaemia.

Another aspect of this invention relates to the furnishing of insulin analogues offering no or little weight gain.

Another aspect of this invention relates to the furnishing of improved lipid profiles. Another aspect of this invention relates to the furnishing of cardioprotective effects.

Another aspect of this invention relates to the furnishing of insulin analogues offering reduced risk of cancer compared with the risk of cancer associated with human insulin.

Another aspect of this invention relates to the furnishing of insulin analogues being liver preferential.

### DEFINITIONS

The term “insulin analogue” as used herein means human insulin wherein one or more amino acid residues have been substituted by other amino acid residues and/or wherein one or more amino acid residues have been deleted and/or wherein one or more amino acid residues have been added.

Herein terms like A1, A2 and A3 etc. indicates the amino acid residue in position 1, 2 and 3 etc., respectively, in the A chain of insulin (counted from the N-terminal end). Similarly, terms like B1, B2 and B3 etc. indicates the amino acid residue in position 1, 2 and 3 etc., respectively, in the B chain of insulin (counted from the N-terminal end). Using the one letter codes for amino acid residues, terms like A21A, A21G and A21Q designates that the amino acid residue in the A21 position is A, G and Q, respectively. Using the three letter codes for amino acid residues, the corresponding expressions are A21 Ala, A21Gly and A21 Gln, respectively.

Herein the terms A(0) and B(0) indicate the positions of the amino acid residues N-terminally to A1 and B1, respectively. The terms A(−1) and B(−1) indicate the positions of the first amino acid residues N-terminally to A(0) and B(0), respectively. Thus A(−2) and B(−2) indicate positions of the amino acid residues N-terminally to A(−1) and B(−1), respectively, A(−3) and B(−3) indicate positions of the amino acid residues N-terminally to A(−2) and B(−2), respectively, and so forth.

Herein, the term amino acid residue is an amino acid from which, formally, a hydroxy group has been removed from a carboxy group and/or from which, formally a hydrogen atom has been removed from an amino group.

### SUMMARY OF THE INVENTION

Briefly, this invention relates to insulin analogues wherein the B25 amino acid residue is His (H) or Asn (N) with the proviso that if the B25 amino acid residue is His, then the B27 amino acid residue is Asp (D) or Glu (E) and the A14 amino acid residue is different from Glu (E).

### DESCRIPTION OF THIS INVENTION

By analysis on the aromatic triplet consisting of B24Phe, B25Phe and B26Tyr together with the bordering B23Gly and B27Thr residues, a whole new class of receptor isoform-selective analogues were discovered. Several analogues with 2-4-fold differences in relative binding affinities for either the A or the B isoform of the insulin receptor compared to human insulin made it possible to identify position B25 as a key determinant for receptor isoform selectivity. Replacement of B25Phe with either Asn resulted in analogues with 2-fold higher relative binding affinities for the B isoform compared to the A isoform of the receptor, whereas the opposite was observed when introducing a Tyr residue at this position. An acidic amino acid residue at position B27 caused an additional 2-fold selective increase in affinity for the receptor B isoform for analogues comprising a B25N mutation. In addition, the combination of B25H with either B27D or B27E was also found to result in B isoform-selective analogues despite the fact that the corresponding single mutation analogues displayed no differences in relative isoform binding affinity. The results demonstrate that one of the mutations at position B25 alone or in combination with a mutation at position B27 in the insulin molecule confers insulin receptor isoform selectivity.

### Production of the Insulin Analogues

The production of polypeptides, e.g. insulins, is well known in the art. An insulin analogue of this invention may for instance be produced by classical peptide synthesis, for example, solid phase peptide synthesis using t-Boc or Fmoc chemistry or other well established techniques, see, for example, Greene and Wuts, “Protective Groups in Organic Synthesis”, John Wiley & Sons, 1999. The insulin analogues of this invention may also be produced by a method which comprises culturing a host cell containing a DNA sequence encoding the analogue and capable of expressing the insulin analogue in a suitable nutrient medium under conditions permitting the expression of the insulin analogue. Hence, briefly, the insulin analogues of this invention are prepared analogously to the preparation of known insulin analogues.

### Indications:

### Diabetes

The term “diabetes” or “diabetes mellitus” includes type 1 diabetes, type 2 diabetes, gestational diabetes (during pregnancy) and other states that cause hyperglycaemia. The term is used for a metabolic disorder in mammals, especially man, in which the pancreas produces insufficient amounts of insulin, or in which the cells of the body fail to respond appropriately to insulin thus preventing cells from absorbing glucose. As a result, glucose builds up in the blood.

Type 1 diabetes, also called insulin-dependent diabetes mellitus (IDDM) and juvenile-onset diabetes, is caused by B-cell destruction, usually leading to absolute insulin deficiency.

Type 2 diabetes, also known as non-insulin-dependent diabetes mellitus (NIDDM) and adult-onset diabetes, is associated with predominant insulin resistance and thus relative insulin deficiency and/or a predominantly insulin secretory defect with insulin resistance.

### Other Indications

In one embodiment, an insulin analogue of this invention is used for the preparation of a medicament for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, stroke, coronary heart disease and other cardiovascular disorders, inflammatory bowel syndrome, dyspepsia and gastric ulcers.

In another embodiment, an insulin analogue of this invention is used as a medicament for delaying or preventing disease progression in type 2 diabetes.

In another embodiment, an insulin analogue of this invention is used as a medicament for decreasing food intake, decreasing (β-cell apoptosis, increasing (β-cell function and (β-cell mass, and/or for restoring glucose sensitivity to p-cells.

In one embodiment of the invention, the insulin analogue of this invention is for use as a medicament for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, coronary heart disease and other cardiovascular disorders, stroke, inflammatory bowel syndrome, dyspepsia and gastric ulcers or for delaying or preventing disease progression in type 2 diabetes or for decreasing food intake, decreasing (β-cell apoptosis, increasing (β-cell function and (β-cell mass, and/or for restoring glucose sensitivity to p-cells, is provided.

In a further embodiment of the invention, a method for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, coronary heart disease and other cardiovascular disorders, stroke, inflammatory bowel syndrome, dyspepsia and gastric ulcers or for delaying or preventing disease progression in type 2 diabetes or for decreasing food intake, decreasing (β-cell apoptosis, increasing (β-cell function and (β-cell mass, and/or for restoring glucose sensitivity to p-cells, the method comprising administering to a patient in need of such treatment an effective amount for such treatment of an insulin analogue of this invention, is provided.

### Diabetes Combination Compounds

The treatment with an insulin analogue of this invention may also be combined with a second or more pharmacologically active substances, for example, selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. Examples of these pharmacologically active substances are: GLP-1 and GLP-1 derivatives and analogues, GLP-2 and GLP-2 derivatives and analogues, Exendin-4 and Exendin-4 derivatives and analogues, amylin and amylin derivatives and analogues, sulphonylureas, biguanides, meglitinides, glucosidase inhibitors, glucagon antagonists, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents as HMG CoA inhibitors (statins), compounds lowering food intake, RXR agonists and agents acting on the ATP-dependent potassium channel of the β-cells; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and a-blockers such as doxazosin, urapidil, prazosin and terazosin; CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists; histamine H3 antagonists, gastrin and gastrin analogues and derivatives.

It should be understood that any suitable combination of an insulin analogue of this invention with one or more of the above-mentioned compounds and optionally one or more further pharmacologically active substances are considered to be within the scope of the present invention.

Use of the Compounds of this Invention
The route of administration may be any route which effectively transports an insulin analogue of this invention to the desired or appropriate place in the body, such as parenterally, for example, subcutaneously, intramuscularly or intraveneously. Alternatively, an insulin analogue of this invention can be administered orally, pulmonary, or nasally.

For parenterally administration, a compound of this invention is formulated analogously with the formulation of known insulins. Furthermore, for parenterally administration, a compound of this invention is administered analogously with the administration of known insulins and the physicians are familiar with this procedure.

Parenteral administration can be performed by means of a syringe, optionally a pen-like syringe. Alternatively, parenteral administration can be performed by means of an infusion pump.

Injectable compositions containing a compound of this invention can be prepared using the conventional techniques of the pharmaceutical industry which involve dissolving and mixing the ingredients as appropriate to give the desired end product. Thus, according to one procedure, a compound of this invention is dissolved in an amount of water which is somewhat less than the final volume of the composition to be prepared. An isotonic agent, a preservative and a buffer is added as required and the pH value of the solution is adjusted, if necessary, using an acid, for example, hydrochloric acid, or a base, for example, aqueous sodium hydroxide, as needed. Finally, the volume of the solution is adjusted with water to give the desired concentration of the ingredients.

More precisely, an insulin preparation of this invention, for example a solution or suspension thereof, may be prepared by dissolving a compound of this invention in an aqueous medium at slightly acidic conditions, for example, in a concentration in the range from about 240 to about 1200 nmole/ml. The aqueous medium is made isotonic, for example, with sodium chloride or glycerol. Furthermore, the aqueous medium may contain zinc ions in a concentration of up to about 20 μg of Zn⁺⁺ per unit of insulin activity, buffers such as acetate and citrate and preservatives such as m-cresol or phenol. The pH value of the solution is adjusted towards neutrality without getting too close to the isoelectric point of the compound of this invention in order to avoid precipitation. The pH value of the final insulin preparation depends upon which compound of this invention is used, the concentration of zinc ions and the concentration of the compound of this invention. The insulin preparation is made sterile, for example, by sterile filtration.

The insulin preparations of this invention are used similarly to the use of the known insulin preparations.

The amount of a compound of this invention to be administered, the determination of how frequently to administer a compound of this invention, and the election of which compound or compounds of this invention to administer, optionally together with another antidiabetic compound, is decided in consultation with a practitioner who is familiar with the treatment of diabetes.

Hence, this invention also relates to a method of treating diabetes, comprising administering an affective amount of a compound of this invention to a patient in need of such treatment.

### PREFERRED FEATURES OF THIS INVENTION

To sum up and supplement the above statements, the features of this invention are as follows:
1. Insulin analogues wherein the B25 amino acid residue is His (H) or Asn (N) with the proviso that if the B25 amino acid residue is His, and the B27 amino acid residue is Asp (D) or Glu (E), then the A14 amino acid residue is different from Glu (E) and with the further proviso that said insulin analogue does not deviate from human insulin in more that 4 positions.
2. Insulin analogues wherein the B25 amino acid residue is His (H) or Asn (N) with the proviso that if the B25 amino acid residue is His, then the B27 amino acid residue is Asp (D) or Glu (E) and the A14 amino acid residue is different from Glu (E).
3. Insulin analogues according to the preceding clause, wherein the B25 amino acid residue is His (H), the B27 amino acid residue is Asp (D) or Glu (E) and the A14 amino acid residue is different from Glu (E).
4. Insulin analogues according to the two preceding clauses wherein not more than 5 of the amino acid residues are different for those present in human insulin on the same positions.
5. Insulin analogues according to clause 1, wherein the B25 amino acid residue is Asn (N).
6. Insulin analogues according to the preceding clause wherein not more than 5 of the amino acid residues are different for those present in human insulin on the same positions.
7. Insulin analogues according to any one of the preceding clauses wherein said analogues are different from A14Q, A22K, B25H, B27E, B29R, desB30 human insulin; A14Q, A22K, B16H, B25H, B27E, B29R, desB30 human insulin; A14Q, A22K, B16E, B25H, B27E, B29R, desB30 human insulin; A14Q, A22K, B16E, B25H, B26G, B27E, B28G, B29R, desB30 human insulin; A14Q, A22K, B16H, B25H, B26G, B27E, B28G, B29R, desB30 human insulin; A14Q, A22K, B25H, B27E, desB30 human insulin; A14Q, A22K, B16H, B25H, B27E, desB30 human insulin; A14Q, A22K, B16E, B25H, B27E, desB30 human insulin; A14Q, A22K, B16E, B25H, B26G, B27E, B28G, desB30 human insulin; A14Q, A22K, B16H, B25H, B26G, B27E, B28G, desB30 human insulin; A14P, A22K, B25H, B27E, B29R, desB30 human insulin; A14P, A22K, B16H, B25H, B27E, B29R, desB30 human insulin; A14P, A22K, B16E, B25H, B27E, B29R, desB30 human insulin; A14P, A22K, B16E, B25H, B26G, B27E, B28G, B29R, desB30 human insulin; A14P, A22K, B16H, B25H, B26G, B27E, B28G, B29R, desB30 human insulin; A14P, A22K, B25H, B27E, desB30 human insulin; A14P, A22K, B16H, B25H, B27E, desB30 human insulin; A14P, A22K, B16E, B25H, B27E, desB30 human insulin; A14P, A22K, B16E, B25H, B26G, B27E, B28G, desB30 human insulin; A14P, A22K, B16H, B25H, B26G, B27E, B28G, desB30 human insulin; A14D, A22K, B25H, B27E, B29R, desB30 human insulin; A14D, A22K, B16H, B25H, B27E, B29R, desB30 human insulin; A14D, A22K, B16E, B25H, B27E, B29R, desB30 human insulin; A14D, A22K, B16E, B25H, B26G, B27E, B28G, B29R, desB30 human insulin; A14D, A22K, B16H, B25H, B26G, B27E, B28G, B29R, desB30 human insulin; A14D, A22K, B25H, B27E, desB30 human insulin; A14D, A22K, B16H, B25H, B27E, desB30 human insulin; A14D, A22K, B16E, B25H, B27E, desB30 human insulin; A14D, A22K, B16E, B25H, B26G, B27E, B28G, desB30 human insulin; and A14D, A22K, B16H, B25H, B26G, B27E, B28G, desB30 human insulin.
8. Insulin analogues according to any one of the preceding clauses wherein said analogues are different from B25N, B27E, desB30 human insulin; A8H, B25N, B27E, desB30 human insulin; B25H, B27E, desB30 human insulin; B1E, B25H, B27E, desB30 human insulin; A8H, B1E, B25H, B27E, desB30 human insulin; A8H, B25H, B27E, desB30 human insulin; B25N, B27D, desB30 human insulin; A8H, B25N, B27D, desB30 human insulin; B25H, B27D, desB309 human insulin; and A8H, B25H, B27D, desB30 human insulin.
9. Insulin analogues according to any one of the preceding clauses wherein said analogues are different from B25N, desB30 human insulin and A21G, B25N, desB30 human insulin.
10. Insulin analogues according to any one of the preceding clauses wherein said analogues are different from A14Q, A22K, B25H, B27E, B29R, desB30 human insulin; A14Q, A22K, B16H, B25H, B27E, B29R, desB30 human insulin; A14Q, A22K, B16E, B25H, B27E, B29R, desB30 human insulin; A14Q, A22K, B16E, B25H, B26G, B27E, B28G, B29R, desB30 human insulin; A14Q, A22K, B16H, B25H, B26G, B27E, B28G, B29R, desB30 human insulin; A14Q, A22K, B25H, B27E, desB30 human insulin; A14Q, A22K, B16H, B25H, B27E, desB30 human insulin; A14Q, A22K, B16E, B25H, B27E, desB30 human insulin; A14Q, A22K, B16E, B25H, B26G, B27E, B28G, desB30 human insulin; A14Q, A22K, B16H, B25H, B26G, B27E, B28G, desB30 human insulin; A14P, A22K, B25H, B27E, B29R, desB30 human insulin; A14P, A22K, B16H, B25H, B27E, B29R, desB30 human insulin; A14P, A22K, B16E, B25H, B27E, B29R, desB30 human insulin; A14P, A22K, B16E, B25H, B26G, B27E, B28G, B29R, desB30 human insulin; A14P, A22K, B16H, B25H, B26G, B27E, B28G, B29R, desB30 human insulin; A14P, A22K, B25H, B27E, desB30 human insulin; A14P, A22K, B16H, B25H, B27E, desB30 human insulin; A14P, A22K, B16E, B25H, B27E, desB30 human insulin; A14P, A22K, B16E, B25H, B26G, B27E, B28G, desB30 human insulin; A14P, A22K, B16H, B25H, B26G, B27E, B28G, desB30 human insulin; A14D, A22K, B25H, B27E, B29R, desB30 human insulin; A14D, A22K, B16H, B25H, B27E, B29R, desB30 human insulin; A14D, A22K, B16E, B25H, B27E, B29R, desB30 human insulin; A14D, A22K, B16E, B25H, B26G, B27E, B28G, B29R, desB30 human insulin; A14D, A22K, B16H, B25H, B26G, B27E, B28G, B29R, desB30 human insulin; A14D, A22K, B25H, B27E, desB30 human insulin; A14D, A22K, B16H, B25H, B27E, desB30 human insulin; A14D, A22K, B16E, B25H, B27E, desB30 human insulin; A14D, A22K, B16E, B25H, B26G, B27E, B28G, desB30 human insulin; A14D, A22K, B16H, B25H, B26G, B27E, B28G, desB30 human insulin; B25N, B27E, desB30 human insulin; A8H, B25N, B27E, desB30 human insulin; B25H, B27E, desB30 human insulin; B1E, B25H, B27E, desB30 human insulin; A8H, B1E, B25H, B27E, desB30 human insulin; A8H, B25H, B27E, desB30 human insulin; B25N, B27D, desB30 human insulin; A8H, B25N, B27D, desB30 human insulin; B25H, B27D, desB309 human insulin; A8H, B25H, B27D, desB30 human insulin; B25N, desB30 human insulin and A21G, B25N, desB30-human insulin.
11. Insulin analogue according to clause 1 or 3, wherein the B25 amino acid residue is Asn (N) and the B27 amino acid residue is different from Lys (K) and Cys (C).
12. Insulin analogue according to any one of the preceding clauses, wherein the A8 amino acid residue is His (H).
13. Insulin analogue according to any one of the preceding clauses wherein the A14 amino acid residue is Tyr (Y).
14. Insulin analogue according to any one of the preceding, possible clause wherein the B27 amino acid residue is E (Glu) or D (Asp).
15. Insulin analogue according to any one of the preceding, possible clause having no amino acid residue in the B30 position (i.e. being a desB30 human insulin analogue).
16. Insulin analogue according to any one of the preceding, possible clause having no amino acid residue in the B29 and B30 positions (i.e. being a desB29, desB30 human insulin analogue).
17. Insulin analogue according to any one of the preceding, possible clause having no amino acid residue in the B28, B29 and B30 positions (i.e. being a desB28, desB29, desB30 human insulin analogue).
18. Insulin analogue according to any one of the preceding, possible clause having no amino acid residue in the B27, B28, B29 and B30 positions (i.e. being a desB27, desB28, desB29, desB30 human insulin analogue).
19. Insulin analogue according to any one of the preceding, possible clause wherein the amino acid in position 26, 27 or 28 of the B chain is Asp or Glu.
20. An insulin analogue according to any one of the preceding clauses to the extend possible which is selected from the group consisting of [A8H, B25N, B27D] human insulin; [A8H, B25N, B27E] human insulin; [A8H, B25N] human insulin; [A8H, B25H, B27D] human insulin; [A8H, B25H, B27E] human insulin; [B25H, B27D] human insulin; [B25H, B27E] human insulin; [B25H] human insulin; [B25N, B27D] human insulin; [B25N, B27E] human insulin; [B25N] human insulin; [A8H, B25N, B27D, desB30] human insulin; [A8H, B25N, B27E, desB30] human insulin; [A8H, B25N, desB30] human insulin; [A8H, B25H, B27D, desB30] human insulin; [A8H, B25H, B27E, desB30] human insulin; [B25H, B27D, desB30] human insulin; [B25H, B27E, desB30] human insulin; [B25H, desB30] human insulin; [B25N, B27D, desB30] human insulin; [B25N, B27E, desB30] human insulin; and [B25N, desB30] human insulin.
21. An insulin analogue according to the preceding clause [A8H, B25N, B27D, desB30] human insulin; [A8H, B25N, B27E, desB30] human insulin; [A8H, B25H, B27D, desB30] human insulin; [A8H, B25H, B27E, desB30] human insulin; [B25H, B27D, desB30] human insulin; [B25H, B27E, desB30] human insulin; [B25N, B27D, desB30] human insulin; [B25N, B27E, desB30] human insulin; and [B25N, desB30] human insulin.
22. An insulin analogue according to any one of the preceding clauses to the extent possible which is [A8H, B25H] human insulin or [A8H, B25H, des B30] human insulin.
23. An insulin analogue according to any one of the preceding clauses to the extend possible which is selected from the group consisting of [A8H, B25N, B27A, desB30] human insulin; [A8H, B25N, B27R, desB30] human insulin; [A8H, B25N, B27N, desB30] human insulin; [A8H, B25N, B27D, desB30] human insulin; [A8H, B25N, B27Q, desB30] human insulin; [A8H, B25N, B27E, desB30] human insulin; [A8H, B25N, B27G, desB30] human insulin; [A8H, B25N, B27H, desB30] human insulin; [A8H, B25N, B271, desB30] human insulin; [A8H, B25N, B27L, desB30] human insulin; [A8H, B25N, B27M, desB30] human insulin; [A8H, B25N, B27F, desB30] human insulin; [A8H, B25N, B27P, desB30] human insulin; [A8H, B25N, B27S, desB30] human insulin; [A8H, B25N, B27W, desB30] human insulin; [A8H, B25N, B27Y, desB30] human insulin and [A8H, B25N, B27V, desB30] human insulin.
24. An insulin analogue according to any one of the preceding clauses to the extent possible which is not any of the following compounds [A8H, B25N, B27D, desB30] human insulin and [A8H, B25N, B27E, desB30] human insulin.
25. Insulin analogue according to any one of the preceding clauses wherein not more than 5 of the amino acid residues are different for those present in human insulin on the same positions.
26. Insulin analogue according to the preceding clause wherein not more than 4 of the amino acid residues are different for those present in human insulin on the same positions.
27. Insulin analogue according to the preceding clause wherein not more than 3 of the amino acid residues are different for those present in human insulin on the same positions.
28. Insulin analogue according to the preceding clause wherein not more than 2 of the amino acid residues are different for those present in human insulin on the same positions.
29. Insulin analogue according to the preceding clause wherein not more than 1 of the amino acid residues are different for those present in human insulin on the same positions.
30. Insulin analogue having an affinity to the IR-B isoform of the insulin receptor which is at least 1.5 times the affinity to the IR-A isoform of the insulin receptor.
31. Insulin analogue according to the preceding clause having an affinity to the IR-B isoform of the insulin receptor which is at least 2 times the affinity to the IR-A isoform of the insulin receptor.
32. Insulin analogue according to the preceding clause having an affinity to the IR-B isoform of the insulin receptor which is at least 2.5 times the affinity to the IR-A isoform of the insulin receptor.
33. Insulin analogue according to the preceding clause having an affinity to the IR-B isoform of the insulin receptor which is at least 3 times the affinity to the IR-A isoform of the insulin receptor.
34. Insulin analogue according to the preceding clause having an affinity to the IR-B isoform of the insulin receptor which is at least 3.5 times the affinity to the IR-A isoform of the insulin receptor.
35. Insulin analogue according to the preceding clause having an affinity to the IR-B isoform of the insulin receptor which is at least 4 times the affinity to the IR-A isoform of the insulin receptor.
36. A method of evaluating whether an insulin analogue is isoform selective, characterized in that one determines the IR-B affinity of the insulin analogue relative to the IR-B affinity of human insulin and determines the IR-A affinity of the insulin analogue relative to the IR-A affinity of human insulin and calculates the ratio between those two, relative affinities.
37. A method of evaluating whether an insulin analogue is liver selective, characterized in that one determines the IR-B affinity of the insulin analogue relative to the IR-B affinity of human insulin and determines the IR-A affinity of the insulin analogue relative to the IR-A affinity of human insulin and calculates the ratio between those two, relative affinities.
38. A compound according to any one of the preceding product clauses, which is any one of the compounds mentioned specifically in the above specification such as in the specific examples, especially any one of examples 1-4 above.
39. A compound according to any one of the preceding product clauses, which is B25N, desB30 human insulin and B25H, B27E, deB30 human insulin.
40. A compound according to any one of the preceding product clauses for use as a medicament or for use in a medicament.
41. A compound according to any one of the preceding product clauses for treating diabetes or the use of a compound according to any one of the preceding product clauses for the preparation of a medicament for the treatment of diabetes.
42. The use of a compound according to any one of the preceding product clauses except the last one for the preparation of a pharmaceutical composition for the treatment of diabetes.
43. A method of treatment of diabetes, the method comprising administering to a subject in need thereof a therapeutically effective amount of a compound according to any one of the preceding product clauses.
44. A method of treatment of diabetes and simultaneously giving a risk of developing cancer which is lower than that associated with human insulin, the method comprising administering to a subject in need thereof a therapeutically effective amount of an insulin analogue according to any one of the preceding clauses.
45. A method according to the preceding clause wherein the insulin analogue is different from any of the insulin analogues mentioned in clause 10 above.
46. Any novel feature or combination of features mentioned in the present specification.

### General Remarks

Combining one or more of the embodiments and/or clauses described herein, optionally also with one or more of the claims below, results in further embodiments and the present invention relates to all possible combinations of said embodiments and claims.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents. The mentioning herein of references is no admission that they constitute prior art.

Herein, the word “comprise” is to be interpreted broadly meaning “include”, “contain” or “comprehend” (EPO guidelines C, III, 4.13).

This invention includes all modifications and equivalents of the subject matter recited in the claims appended hereto as permitted by applicable law.

The following examples are offered by way of illustration, not by limitation.

### Example 1

[ABH, B25N, desB30] human insulin

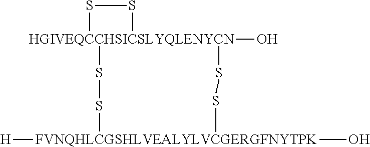

### Example 2

[A8H, B25N, B27E, desB30] human insulin

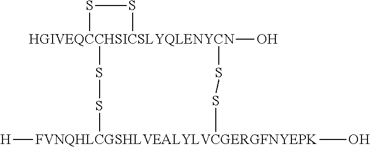

All chemicals and reagents used are of the highest purity available. Immobilized Achromobachter lyticus protease is from Novo Nordisk A/S.

The purification and digestion of the insulin analogue can be made as follows: The insulin precursor from the yeast supernatant is purified and concentrated by cation exchange (Kjeldsen et al., (1998), Prot. Expr. Pur. 14, 309-316). The single-chain insulin precursor is matured into two-chain insulin by digestion with lysine-specific immobilized Achromobachter lyticus protease (hereinafter designated ALP; Kristensen et al., (1997), J. Biol. Chem. 20, 12978-12983). The eluate from the cation exchange chromatography step containing the insulin precursor is diluted with water to an ethanol concentration of 15-20%. Sodium glutamate is added to a concentration of 15 mM and pH is adjusted to 9.7 by NaOH. Immobilized ALP (4 gram/L) is added in a proportion of 1:100 (volume:volume) and digestion is allowed to proceed with mild stirring in room temperature overnight. The digestion reaction is analyzed by analytical LC on a Waters Acquity Ultra-Performance Liquid Chromatography system using a C18 column and the molecular weight is confirmed by matrix-assisted laser desorption ionization time-of-flight mass spectrometry (Bruker Daltonics Autoflex II TOF/TOF). The immobilized A. lyticus protease is removed by filtration with a 0.2 μm filter. The two-chain insulin molecule is purified by reversed phase HPLC (Waters 600 system) on a C18 column using an acetonitrile gradient. The desired A8H, B25N, B27E, desB30 human insulin (5.11 g) is recovered by lyophilization. Purity is determined by analytical LC on a Waters Acquity Ultra-Performance Liquid Chromatography system using a C18 column, and the molecular weight is confirmed by matrix-assisted laser desorption ionization time-of-flight mass spectrometry.

### Example 3

[A8H, B25H, B27D, desB30] human insulin

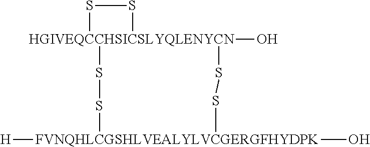

### Example 4

[A8H, B25H, B27E, desB30] human insulin

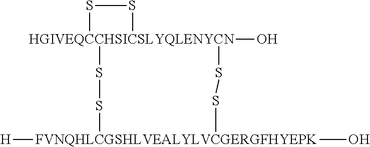

### Example 5

[A8H, B25N, B27D, desB30] human insulin

### Example 6

[B25H, B27E, desB30] human insulin

### Example 7

[B25N, desB30] human insulin

### Example 8

Insulin Receptor Binding Assay (on solubilised insulin receptor)

The affinity of the insulins of the invention for the human insulin receptor is determined by a Scintillation Proximity Assay (SPA) (according to Glendorf et al. (2008), Biochemistry, 47, 4743-4751). Competition binding experiments were performed in 96-well plates (polystyrene Optiplate-96, PerkinElmer) on an Eppendorf epMotion 5075 robot using solubilized human IR (holoreceptor) semipurified by wheat germ agglutinin purification from baby hamster kidney (BHK) cells, which were stably transfected with the pZem vector containing the human IR-A or IR-B insert. Assays were initiated by making dilution series (eight dilutions, 5-fold each, first dilution 43-fold) of yeast supernatant containing the two-chain analogue and a human insulin standard. A reagent mix consisting of SPA beads (SPA PVT Antibody-Binding Beads, Anti-Mouse Reagent Cat. No. RPNQ0017, GE Healthcare) resuspended in binding buffer, anti-IR monoclonal mouse antibody (83-7), solubilized human IR (hIR-A or hIR-B), and [¹²⁵I]A14Tyr-labelled insulin was added to the dilution series of the appropriate samples. The final concentration of [¹²⁵I]A14Tyr-labelled insulin was 7.5 pM, and the buffer consisted of 100 mM HEPES (pH 7.8), 100 mM NaCI, 10 mM MgSO4, and 0.025% (v/v) Tween 20. Plates were incubated with gentle shaking for 24 h at room temperature, centrifuged for 2 minutes at 2000 rpm, and counted in a TopCount NXT for 3 min/well. Data from the SPA were analyzed according to the four-parameter logistic model (Vølund, A., (1978), Biometrics, 34, 357-365.) and the affinities of the insulin analogues expressed relative to that of human insulin.

Preparation of Monoclonal mIR Antibodies

Specific antibodies (F12 or 83-7) are produced by monoclonal technique: RBF mice are immunized by injecting 50 μg of purified mIR in FCA subcutaneously followed by two injections with 20 μg of mIR in FIA. High responder mice are boosted intravenously with 25 μg of mIR and the spleens are harvested after 3 days. Spleen cells are fused with the myeloma Fox cell line (Köhler, G & Milstein C. (1976), European J. Immunology, 6:511-19; Taggart RT et al (1983), Science 219:1228-30). Supernatants are screened for antibody production in a mIR specific ELISA. Positive wells are cloned and tested in Western blotting.
In the following tables, the affinities of the desB30 insulin analogues are expressed relative to human insulin in an assay using either hIR-A or hIR-B. The IR affinities were assessed using insulin analogues directly in yeast supernatant. DesB30 human insulin displays a relative affinity of 98 and 100% for hIR-A and hIR-B, respectivtly

In the following table, the data given in the column with he heading “hIR-A” is the ratio between the hIR-A affinity of the compound tested in relation to the hIR-A affinity of human insulin, the data given in the column with he heading “hIR-B” is the ratio between the hIR-B affinity of the compound tested in relation to the hIR-B affinity of human insulin and the data given in the column with the heading “Ratio B/A” is the ratio between the figure given in the columns with the headings “hIR-B” and “hIR-A”, respectively.

| Example | hIR-A; [% of HI] | hIR-B; [% of HI]; (%) | Ratio B/A |
|---|---|---|---|
| 1 | 22 ± 4 | 44 ± 5 | 2.0 |
| 2 | 15 ± 3 | 57 ± 3 | 3.8 |
| 3 | 43 ± 4 | 82 ± 7 | 1.9 |
| 4 | 67 ± 3 | 140 ± 5 | 2.1 |
| 5 | 10 ± 1 | 38 ± 4 | 3.8 |
| 6 | 13 ± 1 | 30 ± 5 | 2.3 |
| 7 | 4 ± 0.2 | 8 ± 0.7 | 2.0 |

As appears from this table, the relative affinity for hIR-B is up to 4-fold higher than the relative affinity for hIR-A compared to human insulin.

### Example 9

The compounds appearing in the following table were tested as described in Example 8. In this table, the data in the column with the heading “Ratio B/A” are calculated as mentioned in example 8.

| Compound tested | Ratio B/A |
|---|---|
| [A8H, B25N, B27A, desB30] human insulin | 2.9 |
| [A8H, B25N, B27R, desB30] human insulin | 2.2 |
| [A8H, B25N, B27N, desB30] human insulin | 2.2 |
| [A8H, B25N, B27D, desB30] human insulin | 3.5 |
| [A8H, B25N, B27Q, desB30] human insulin | 2.2 |
| [A8H, B25N, B27E, desB30] human insulin | 4.6 |
| [A8H, B25N, B27G, desB30] human insulin | 2.8 |
| [A8H, B25N, B27H, desB30] human insulin | 2.4 |
| [A8H, B25N, B27I, desB30] human insulin | 2.1 |
| [A8H, B25N, B27L, desB30] human insulin | 2.0 |
| [A8H, B25N, B27M, desB30] human insulin | 3.0 |
| [A8H, B25N, B27F, desB30] human insulin | 2.6 |
| [A8H, B25N, B27P, desB30] human insulin | 2.6 |
| [A8H, B25N, B27S, desB30] human insulin | 3.0 |
| [A8H, B25N, B27W, desB30] human insulin | 2.2 |
| [A8H, B25N, B27Y, desB30] human insulin | 3.6 |
| [A8H, B25N, B27V, desB30] human insulin | 2.6 |

As appears from this table, the relative affinity for hIR-B is in the range 2 to 4.6 times the relative affinity for hIR-A, compared to human insulin.

### Sequence Listings

SEQ ID NO:1 is the A chain of examples 1-4, SEQ ID NO:2 is the B chain of example 1, SEQ ID NO:3 is the B chain of example 2, SEQ ID NO:4 is the B chain of example 3 and SEQ ID NO:5 is the B chain of example 4.

1. An insulin analogue wherein the B25 amino acid residue is His (H) or Asn (N) with the proviso that if the B25 amino acid residue is His, then the B27 amino acid residue is Asp (D) or Glu (E) and the A14 amino acid residue is different from Glu (E), and
provided said analogues are not
A14Q, A22K, B25H, B27E, B29R, desB30 human insulin;
A14Q, A22K, B16H, B25H, B27E, B29R, desB30 human insulin;
A14Q, A22K, B16E, B25H, B27E, B29R, desB30 human insulin;
A14Q, A22K, B16E, B25H, B26G, B27E, B28G, B29R, desB30 human insulin;
A14Q, A22K, B16H, B25H, B26G, B27E, B28G, B29R, desB30 human insulin;
A14Q, A22K, B25H, B27E, desB30 human insulin;
A14Q, A22K, B16H, B25H, B27E, desB30 human insulin;
A14Q, A22K, B16E, B25H, B27E, desB30 human insulin;
A14Q, A22K, B16E, B25H, B26G, B27E, B28G, desB30 human insulin;
A14Q, A22K, B16H, B25H, B26G, B27E, B28G, desB30 human insulin;
A14P, A22K, B25H, B27E, B29R, desB30 human insulin;
A14P, A22K, B16H, B25H, B27E, B29R, desB30 human insulin;
A14P, A22K, B16E, B25H, B27E, B29R, desB30 human insulin;
A14P, A22K, B16E, B25H, B26G, B27E, B28G, B29R, desB30 human insulin;
A14P, A22K, B16H, B25H, B26G, B27E, B28G, B29R, desB30 human insulin;
A14P, A22K, B25H, B27E, desB30 human insulin;
A14P, A22K, B16H, B25H, B27E, desB30 human insulin;
A14P, A22K, B16E, B25H, B27E, desB30 human insulin;
A14P, A22K, B16E, B25H, B26G, B27E, B28G, desB30 human insulin;
A14P, A22K, B16H, B25H, B26G, B27E, B28G, desB30 human insulin;
A14D, A22K, B25H, B27E, B29R, desB30 human insulin;
A14D, A22K, B16H, B25H, B27E, B29R, desB30 human insulin;
A14D, A22K, B16E, B25H, B27E, B29R, desB30 human insulin;
A14D, A22K, B16E, B25H, B26G, B27E, B28G, B29R, desB30 human insulin;
A14D, A22K, B16H, B25H, B26G, B27E, B28G, B29R, desB30 human insulin;
A14D, A22K, B25H, B27E, desB30 human insulin;
A14D, A22K, B16H, B25H, B27E, desB30 human insulin;
A14D, A22K, B16E, B25H, B27E, desB30 human insulin;
A14D, A22K, B16E, B25H, B26G, B27E, B28G, desB30 human insulin;
A14D, A22K, B16H, B25H, B26G, B27E, B28G, desB30 human insulin;
B25N, B27E, desB30 human insulin;
A8H, B25N, B27E, desB30 human insulin;
B25H, B27E, desB30 human insulin;
B1E, B25H, B27E, desB30 human insulin;
A8H, B1E, B25H, B27E, desB30 human insulin;
A8H, B25H, B27E, desB30 human insulin;
B25N, B27D, desB30 human insulin;
A8H, B25N, B27D, desB30 human insulin;
B25H, B27D, desB30 human insulin;
A8H, B25H, B27D, desB30 human insulin;
B25N, desB30 human insulin, and A21G, B25N, desB30-human insulin.

2. (canceled)

3. An insulin analogue according to claim 1, wherein the B25 amino acid residue is His (H), the B27 amino acid residue is Asp (D) or Glu (E) and the A14 amino acid residue is different from Glu (E).

4. An insulin analogue according to claim 1, wherein the B25 amino acid residue is Asn (N).

5. Insulin analogue according to claim 1 having no amino acid residue in the B29 and B30 positions (i.e. being a desB29, desB30 human insulin analogue).

6. Insulin analogue according to claim 1 having no amino acid residue in the B28, B29 and B30 positions (i.e. being a desB28, desB29, desB30 human insulin analogue).

7. Insulin analogue according to claim 1 having no amino acid residue in the B27, B28, B29 and B30 positions (i.e. being a desB27, desB28, desB29, desB30 human insulin analogue).

8. Insulin analogue according to claim 1 having no amino acid residue in the B27, B28, B29 and B30 positions (i.e. being a desB27, desB28, desB29, desB30 human insulin analogue), preferably an insulin analogue according to any one of the preceding, possible claims wherein the amino acid in position 26, 27 or 28 of the B chain is Asp or Glu.

9. An insulin analogue according to claim 1 which is selected from the group consisting of [A8H, B25N, B27D] human insulin; [A8H, B25N, B27E] human insulin; [A8H, B25N] human insulin; [A8H, B25H, B27D] human insulin; [A8H, B25H, B27E] human insulin; [B25H, B27D] human insulin; [B25H, B27E] human insulin; [B25H] human insulin; [B25N, B27D] human insulin; [B25N, B27E] human insulin; [B25N] human insulin; [A8H, B25N, B27D, desB30] human insulin; [A8H, B25N, B27E, desB30] human insulin; [A8H, B25N, desB30] human insulin; [A8H, B25H, B27D, desB30] human insulin; [A8H, B25H, B27E, desB30] human insulin; [B25H, B27D, desB30] human insulin; [B25H, B27E, desB30] human insulin; [B25H, desB30] human insulin; [B25N, B27D, desB30] human insulin; [B25N, B27E, desB30] human insulin; and [B25N, desB30] human insulin.

10. An insulin analogue according to claim 1, which analogue is [A8H, B25H] human insulin or [A8H, B25H, des B30] human insulin.

11. An insulin analogue according to claim 1 which is selected from the group consisting of [A8H, B25N, B27A, desB30] human insulin; [A8H, B25N, B27R, desB30] human insulin; [A8H, B25N, B27N, desB30] human insulin; [A8H, B25N, B27D, desB30] human insulin; [A8H, B25N, B27Q, desB30] human insulin; [A8H, B25N, B27E, desB30] human insulin; [A8H, B25N, B27G, desB30] human insulin; [A8H, B25N, B27H, desB30] human insulin; [A8H, B25N, B27I, desB30] human insulin; [A8H, B25N, B27L, desB30] human insulin; [A8H, B25N, B27M, desB30] human insulin; [A8H, B25N, B27F, desB30] human insulin; [A8H, B25N, B27P, desB30] human insulin; [A8H, B25N, B27S, desB30] human insulin; [A8H, B25N, B27W, desB30] human insulin; [A8H, B25N, B27Y, desB30] human insulin and [A8H, B25N, B27V, desB30] human insulin.

12. A method of treatment of diabetes, the method comprising administering to a subject in need thereof a therapeutically effective amount of a compound according to claim 1.

13. A method of treatment of diabetes and simultaneously giving a risk of developing cancer which is lower that that associated with human insulin, the method comprising administering to a subject in need thereof a therapeutically effective amount of an insulin analogue according to claim 1.

14. (canceled)

15. (canceled)
